SEQUENCE LISTING (62 sequences)

SEQ ID NO: 1 - DNA, 14 bp, Artificial
  misc_feature: detection primer oligonucleotide segment  for the B-RAF mutation G1388A
ggacaaagaattga

SEQ ID NO: 2 - DNA, 14 bp, Artificial
  misc_feature: detection primer oligonucleotide segment  for the B-RAF mutation G1388T
ggacaaagaattgt

SEQ ID NO: 3 - DNA, 14 bp, Artificial
  misc_feature: detection primer oligonucleotide segment for the B-RAF mutation G 1394C
agaattggatctgc

SEQ ID NO: 4 - DNA, 14 bp, Artificial
  misc_feature: detection primer oligonucleotide segment  for the B-RAF mutation G1394A
agaattggatctga

SEQ ID NO: 5 - DNA, 14 bp, Artificial
  misc_feature: detection primer oligonucleotide segment for the B-RAF mutation G 1394T
agaattggatctgt

SEQ ID NO: 6 - DNA, 14 bp, Artificial
  misc_feature: detection primer oligonucleotide segment for the B-RAF mutation G 1403C
tctggatcatttgc

SEQ ID NO: 7 - DNA, 14 bp, Artificial
  misc_feature: detection primer oligonucleotide segment for the B-RAF mutation G 1403A
tctggatcatttga

SEQ ID NO: 8 - DNA, 14 bp, Artificial
  misc_feature: detection primer oligonucleotide segment for the B-RAF mutation G 1753A
tatatttcttcata

SEQ ID NO: 9 - DNA, 14 bp, Artificial
  misc_feature: detection primer oligonucleotide segment for the B-RAF mutation T 1782G
aaataggtgatttg

SEQ ID NO: 10 - DNA, 14 bp, Artificial
  misc_feature: detection primer oligonucleotide segment for the B-RAF mutation G 1783C
aataggtgattttc

SEQ ID NO: 11 - DNA, 14 bp, Artificial
  misc_feature: detection primer oligonucleotide segment for the B-RAF mutation C 1786G
aggtgattttggtg

SEQ ID NO: 12 - DNA, 14 bp, Artificial
  misc_feature: detection primer oligonucleotide segment for the B-RAF mutation T 1787G
ggtgattttggtcg

SEQ ID NO: 13 - DNA, 14 bp, Artificial
  misc_feature: detection primer oligonucleotide segment for the B-RAF mutation T 1796A
ggtctagctacaga

SEQ ID NO: 14 - DNA, 14 bp, artificial
  misc_feature: detection primer oligonucleotide segment for the B-RAF mutation T G1796-97AT
gtctagctacagat

SEQ ID NO: 15 - DNA, 16 bp, Artificial
  misc_feature: detection primer oligonucleotide segment for the B-RAF mutation G 1388A
tgggacaaagaattga

SEQ ID NO: 16 - DNA, 16 bp, Artificial
  misc_feature: detection primer oligonucleotide segment for the B-RAF mutation G 1388T
tgggacaaagaattgt

SEQ ID NO: 17 - DNA, 16 bp, Artificial
  misc_feature: detection primer oligonucleotide segment for the B-RAF mutation G 1394C
aaagaattggatctgc

SEQ ID NO: 18 - DNA, 16 bp, Artificial
  misc_feature: detection primer oligonucleotide segment for the B-RAF mutation G 1394A
aaagaattggatctga

SEQ ID NO: 19 - DNA, 16 bp, Artificial
  misc_feature: detection primer oligonucleotide segment for the B-RAF mutation G 1394T
aaagaattggatctgt

SEQ ID NO: 20 - DNA, 16 bp, Artificial
  misc_feature: detection primer oligonucleotide segment for the B-RAF mutation G 1403C
gatctggatcatttgc

SEQ ID NO: 21 - DNA, 16 bp, artificial
  misc_feature: detection primer oligonucleotide segment for the B-RAF mutation G 1403A
gatctggatcatttga

SEQ ID NO: 22 - DNA, 16 bp, Artificial
  misc_feature: detection primer oligonucleotide segment for the B-RAF mutation G 1753A
aatatatttcttcata

SEQ ID NO: 23 - DNA, 16 bp, Artificial
  misc_feature: detection primer oligonucleotide segment for the B-RAF mutation T 1782G
aaaaataggtgatttg

SEQ ID NO: 24 - DNA, 16 bp, Artificial
  misc_feature: detection primer oligonucleotide segment for the B-RAF mutation G 1783C
aaaataggtgattttc

SEQ ID NO: 25 - DNA, 16 bp, Artificial
  misc_feature: detection primer oligonucleotide segment for the B-RAF mutation C 1786G
ataggtgattttggtg

SEQ ID NO: 26 - DNA, 16 bp, Artificial
  misc_feature: detection primer oligonucleotide segment for the B-RAF mutation T 1787G
taggtgattttggtcg

SEQ ID NO: 27 - DNA, 16 bp, Artificial
  misc_feature: detection primer oligonucleotide segment for the B-RAF mutation T 1796A
ttggtctagctacaga

SEQ ID NO: 28 - DNA, 16 bp, Artificial
  misc_feature: detection primer oligonucleotide segment for the B-RAF mutation T G1796-97AT
tggtctagctacagat

SEQ ID NO: 29 - DNA, 20 bp, Artificial
  misc_feature: detection primer oligonucleotide segment for the B-RAF mutation G 1388A
acagtgggacaaagaattga

SEQ ID NO: 30 - DNA, 20 bp, Artificial
  misc_feature: detection primer oligonucleotide segment for the B-RAF mutation G 1388T
acagtgggacaaagaattgt

SEQ ID NO: 31 - DNA, 20 bp, Artificial
  misc_feature: detection primer oligonucleotide segment for the B-RAF mutation G 1394C
ggacaaagaattggatctgc

SEQ ID NO: 32 - DNA, 20 bp, artificial
  misc_feature: detection primer oligonucleotide segment for the B-RAF mutation G 1394A
ggacaaagaattggatctga

SEQ ID NO: 33 - DNA, 20 bp, Artificial
  misc_feature: detection primer oligonucleotide segment for the B-RAF mutation G 1394T
ggacaaagaattggatctgt

SEQ ID NO: 34 - DNA, 20 bp, Artificial
  misc_feature: detection primer oligonucleotide segment for the B-RAF mutation G 1403C
attggatctggatcatttgc

SEQ ID NO: 35 - DNA, 20 bp, Artificial
  misc_feature: detection primer oligonucleotide segment for the B-RAF mutation G 1403A
attggatctggatcatttga

SEQ ID NO: 36 - DNA, 23 bp, Artificial
  misc_feature: detection primer oligonucleotide segment for the B-RAF mutation G 1753A
gagtaataatatatttcttcata

SEQ ID NO: 37 - DNA, 20 bp, Artificial
  misc_feature: detection primer oligonucleotide segment for the B-RAF mutation T 1782G
cagtaaaaataggtgatttg

SEQ ID NO: 38 - DNA, 21 bp, Artificial
  misc_feature: detection primer oligonucleotide segment for the B-RAF mutation G 1783C
cagtaaaaataggtgattttc

SEQ ID NO: 39 - DNA, 22 bp, Artificial
  misc_feature: detection primer oligonucleotide segment for the B-RAF mutation C 1786G
gtaaaaataggtgattttggtg

SEQ ID NO: 40 - DNA, 23 bp, Artificial
  misc_feature: detection primer oligonucleotide segment for the B-RAF mutation T 1787G
gtaaaaataggtgattttggtcg

SEQ ID NO: 41 - DNA, 20 bp, Artificial
  misc_feature: detection primer oligonucleotide segment for the B-RAF mutation T 1796A
gattttggtctagctacaga

SEQ ID NO: 42 - DNA, 21 bp, Artificial
  misc_feature: detection primer oligonucleotide segment for the B-RAF mutation T G1796-97AT
gattttggtctagctacagat

SEQ ID NO: 43 - DNA, 23 bp, Artificial
  misc_feature: second primer oligonucleotide segment for the B-RAF mutation G1388A
tgtcaccacattacatacttacc

SEQ ID NO: 44 - DNA, 23 bp, Artificial
  misc_feature: second primer oligonucleotide segment for the B-RAF mutation G1388T
tgtcaccacattacatacttacc

SEQ ID NO: 45 - DNA, 23 bp, Artificial
  misc_feature: second primer oligonucleotide segment for the B-RAF mutation G1394C
tgtcaccacattacatacttacc

SEQ ID NO: 46 - DNA, 23 bp, Artificial
  misc_feature: second primer oligonucleotide segment for the B-RAF mutation G1394A
tgtcaccacattacatacttacc

SEQ ID NO: 47 - DNA, 23 bp, Artificial
  misc_feature: second primer oligonucleotide segment for the B-RAF mutation G1394A
tgtcaccacattacatacttacc

SEQ ID NO: 48 - DNA, 23 bp, Artificial
  misc_feature: second primer oligonucleotide segment for the B-RAF mutation G1403C
tgtcaccacattacatacttacc

SEQ ID NO: 49 - DNA, 23 bp, Artificial
  misc_feature: second primer oligonucleotide segment for the B-RAF mutation G1403A
tgtcaccacattacatacttacc

SEQ ID NO: 50 - DNA, 21 bp, Artificial
  misc_feature: detection primer oligonucleotide segment for the B-RAF mutation G1753A
gactttctagtaactcagcag

SEQ ID NO: 51 - DNA, 21 bp, Artificial
  misc_feature: second primer oligonucleotide segment for the B-RAF mutation T1782G
gactttctagtaactcagcag

SEQ ID NO: 52 - DNA, 21 bp, Artificial
  misc_feature: second primer oligonucleotide segment for the B-RAF mutation G1783C
gactttctagtaactcagcag

SEQ ID NO: 53 - DNA, 21 bp, Artificial
  misc_feature: second primer oligonucleotide segment for the B-RAF mutation C1786G
gactttctagtaactcagcag

SEQ ID NO: 54 - DNA, 21 bp, Artificial
  misc_feature: second primer oligonucleotide segment for the B-RAF mutation T1787G
gactttctagtaactcagcag

SEQ ID NO: 55 - DNA, 21 bp, Artificial
  misc_feature: second primer oligonucleotide segment for the B-RAF mutation T1796A
gactttctagtaactcagcag

SEQ ID NO: 56 - DNA, 21 bp, Artificial
  misc_feature: second primer oligonucleotide segment for the B-RAF mutation TG1796-97AT
gactttctagtaactcagcag

SEQ ID NO: 57 - DNA, 23 bp, Artificial
  misc_feature: detection primer oligonucleotide segment for the B-RAF mutation T1796-97A
ggacccactccatcgagatttct

SEQ ID NO: 58 - DNA, 21 bp, Artificial
  misc_feature: second primer oligonucleotide segment for the B-RAF mutation T1796-97A
cataatgcttgctctgatagg

SEQ ID NO: 59 - DNA, 22 bp, Artificial
  misc_feature: detection primer oligonucleotide segment for the B-RAF mutation T1796-97A
gacccactccatcgagatttct

SEQ ID NO: 60 - DNA, 21 bp, Artificial
  misc_feature: detection primer oligonucleotide segment for the B-RAF mutation T1796-97A
acccactccatcgagatttct

SEQ ID NO: 61 - DNA, 20 bp, Artificial
  misc_feature: detection primer oligonucleotide segment for the B-RAF mutation T1796-97A
cccactccatcgagatttct

SEQ ID NO: 62 - DNA, 19 bp, Artificial
  misc_feature: detection primer oligonucleotide segment for the B-RAF mutation T1796-97A
ccactccatcgagatttct

(19) United States
(12) Patent Application Publication
(10) Pub. No.: US 2006/0252041 A1
(43) Pub. Date: Nov. 9, 2006

(54) PCR-based diagnostic method of detecting a mutation in the b-raf gene

(76) Inventor: David Bryant Batt, Bridgewater, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/528,915

(22) PCT Filed: Sep. 25, 2003
(86) PCT No.: PCT/EP03/10675

§ 371 Date: Jan. 5, 2006

Related U.S. Application Data
(60) Provisional application No. 60/413,709, filed on Sep. 26, 2002.

(51) Int. Cl. C12Q 1/68; C07H 21/04

(52) U.S. Cl. 435/6; 536/24.3

(57) ABSTRACT

This disclosure relates to a PCR based method for detecting a mutation in the B-RAF gene which involves utilizing a primer that is complementary to the mutated base(s) and detecting the mutation by determining whether amplification of the DNA occurs.

### SUMMARY OF THE INVENTION

This invention relates to a convenient polymerase chain reaction (PCR) based method for detecting a specific mutation in nucleic acids, particularly genomic DNA.

### BACKGROUND OF THE INVENTION

It has been postulated that some cancers and other proliferative disorders are caused by mutations that transform a normal cell in a manner which promotes proliferation or interferes with cell differentiation or normal cell death. Such mutations may, for example, activate genes that promote cell growth, interfere with cell differentiation or apoptosis pathways, produce enzymes or other factors having increased or decreased activity, and the like. The presence or absence of a particular mutation may be the basis for decisions relating to proper treatment of the disorder. Thus, the ability to detect such mutations can be of great importance for proper treatment of such disorders.

The present invention relies on PCR as the basis for a diagnostic method for determining whether a specific mutation is present in a particular segment of nucleic acid. PCR is a well-known technique for the in vitro amplification of a segment of nucleic acid that lies between two regions of known sequence. Generally, a template nucleic acid is denatured, for example, by heat, and are then permitted to anneal to two oligonucleotide primers. The primers are complementary to sequences on opposite strands of the template nucleic acid and flank the nucleic acid segment to be amplified. Upon exposure to an appropriate nucleic acid polymerase, for example a DNA polymerase, under conditions known to those of skill in the art, two new copies of the desired nucleic acid segment are prepared. The cycle is repeated several times and the number of copies of the desired nucleic acid segment increases, theoretically by 2ⁿ fold, where n is the number of cycles. As is apparent, after several cycles many new copies of the desired nucleic acid segment are prepared.

According to the present invention, the 3′ end of one of the primers (hereinafter “detection primer”) is complementary to a suspected mutation on a first strand of the template nucleic acid and the second primer is complementary to a segment of the opposite strand of the template nucleic acid that is sufficiently upstream or downstream of the mutation such that the amplified product is detectable if it is produced by PCR. The conditions for carrying out the PCR are selected such that the PCR proceeds only when the 3′ end of the detection primer is complementary to the first strand of the template nucleic acid. Thus, if the 3′ end of the detection primer is complementary to the mutated base, amplification of the nucleic acid segment by PCR indicates that the mutation is present in the template nucleic acid.

### DETAILED DESCRIPTION OF THE INVENTION

In a first aspect, the present invention is a method for detecting a mutation in a nucleic acid, which comprises:

(a) attempting to amplify a segment of the nucleic acid containing the mutation by a PCR utilizing a nucleic acid polymerase without exonuclease activity in the presence of a detection primer and a second primer, wherein the 3′ end of the detection primer is known to be complementary to the mutated base on a first strand of the nucleic acid and the second primer is complementary to a segment of the opposite strand of the nucleic acid and selected such that a detectable amplification product will be produced if the PCR occurs; and

(b) detecting whether the nucleic acid segment is amplified.

Preferably, the nucleic acid is DNA, such as genomic DNA. Thus, this aspect of the invention more particularly relates to a method for detecting a specific mutation in a DNA of known sequence, which comprises:

(a) subjecting a segment of the DNA containing the mutation to amplification by a PCR utilizing a DNA polymerase without 3′→5′ exonuclease activity in the presence of a detection primer and a second primer, wherein 3′ end of the detection primer is known to be complementary to the mutated base on a first strand of the DNA segment and the second primer is complementary to a segment of the opposite DNA strand and selected such that detectable amplification of the nucleic acid segment comprising the mutated base can occur; and

(b) detecting whether the segment of the DNA is amplified.

The primers are oligonucleotides that are complementary to sequences on opposite strands of the template nucleic acid and flank the nucleic acid segment to be amplified. The primers should be at least about 14 nucleotides in length, and preferably about 16-24 nucleotides in length, for example, 20-24 nucleotides in length. The basis of this aspect of the invention is the use of a detection primer having a 3′ end which is known to be complementary with the mutated base on the nucleic acid segment, such as a DNA segment, thought to contain the mutation. The PCR is carried out under conditions whereby the no elongation occurs at the 3′ end of the detection primer if the base at the 3′ end is not complementary to the base present at the mutation point on the template nucleic acid strand. Thus, no significant amount of amplification of the nucleic acid segment will take place unless the 3′ end of the detection primer is complementary to the base present at the point of the mutation in the template nucleic acid. Accordingly, if the 3′ end of the detection primer is complementary to the mutation, amplification of the nucleic acid segment indicates that the mutation is present.

The nucleic acid segment that is amplified according to the invention is defined by the two primers because the PCR will amplify the portion of the template nucleic acid segment which is flanked by the two primers. Therefore, the second primer is selected to be complementary to the opposite strand of the nucleic acid and sufficiently upstream or downstream of the mutation such that the amplified product is readily detectable. In general, the nucleic acid segment to be amplified should include at least about 70 base pairs. It is preferred for the nucleic acid segment to be amplified to be at least 70 base pairs in length, but not more than 6,000 base pairs in length, for example, about 100-1,000 or 150-500 base pairs in length. In each instance, the nucleic acid segment includes the mutation that is to be detected.

According to the inventive method, the PCR is carried out under conditions whereby no significant amplification will occur if the base at the 3′ end of the detection primer is not complementary to the counterpart base on the template nucleic acid. Such conditions are generally standard but require the use of a nucleic acid polymerase without 3′→5′ exonuclease activity under the conditions being used. Such nucleic acid polymerases without 3′→5′ exonuclease activity are known to those of skill in the art and are commercially available. They include, for example, Taq, Vent (exo-) [New England Biolabs], Deep Vent (exo-) [New England Biolabs], 9° N polymerases [New England Biolabs] and MasterAmp™ AmpliTherm™ [Epicentre] polymerases, all of which are commercially available. It is preferred for the nucleic acid to be DNA and the DNA polymerase to be a DNA polymerase selected from Taq, VENT (exo-), DEEP VENT (exo-), 9° N and MASTERAMP AMPLITHERM DNA polymerases, especially Taq DNA polymerase.

Generally, according to the inventive method, from about 20-40 cycles of PCR are carried out. Preferably, from about 25-35 cycles, especially 35 cycles of PCR are carried out.

Whether amplification of the segment of nucleic acid occurs is detected by methods known to those of skill. Such detection methods especially include gel electrophoresis with DNA staining by methods, such as ethidium bromide.

According to the inventive method, amplification will occur only if the base at the 3′ end of the detection primer is complementary to the corresponding base in the template nucleic acid. Thus, if 3′ end of the detection primer is complementary to the mutation, the presence of an amplification product indicates that the mutation is present in the template nucleic acid strand and the absence of an amplification product indicates that the template nucleic acid does not contain the mutation.

Thus, the present invention includes a method for detecting a specific mutation in a nucleic acid, which comprises:

(a) attempting to amplify a segment of the nucleic acid containing the mutation by a PCR utilizing a nucleic acid polymerase without 3′→5′ exonuclease activity in the presence of a detection primer and a second primer, wherein the 3′ end of the detection primer is known to be complementary to the mutated base at the point of mutation on a first strand of the nucleic acid and the second primer is complementary to a segment of the opposite strand of the nucleic acid and selected such that a detectable amplification product will be produced if the PCR occurs;

(b) detecting whether the nucleic acid segment is amplified; and

(c) indicating that the mutation is present if the nucleic acid segment is amplified.

One of skill in the art understand the criteria used to select a suitable second primer for PCR. In general, the second primer is complementary to a segment of the opposite DNA strand and sufficiently upstream or downstream of the mutation such that if the PCR occurs, the amplified product will include the mutation and contain enough nucleotides to be readily detectable. Preferably, the second primer is selected such that it is at least about 14 nucleotides in length, and preferably about 16-24 nucleotides in length, for example, 20-24 nucleotides in length, the amplification product will contain at least about 70 base pairs, for example, 70-6,000 base pairs, preferably 100-1,000 base pairs, for example, 150-500 base pairs, and preferably has a melting point similar to that of the detection primer.

Another aspect of this invention relates to the detection of mutations in the B-RAF gene. B-RAF is a serine/threonine kinase that functions in the RAS-RAF-MEK-ERK kinase pathway. The nucleotide sequence of the human B-RAF gene is known. It has recently been reported that B-RAF is commonly activated by one of several somatic point mutations in human cancer, including 59% of the melanoma cell lines tested. See Davies et al., Nature, Vol. 417, pp. 949-954 (2002). The ability to detect these mutations in the B-RAF gene of cancer patients will lead to rational treatment options that include, for example, treatment with compounds that inhibit B-RAF kinase or limit expression of the mutant kinase.

According to this aspect of the present invention, mutations in the B-RAF gene are detected by the PCR based method described above. Thus, the present invention further relates to a method for detecting a specific mutation in the B-RAF gene, which comprises:

(a) subjecting a segment of the B-RAF gene containing the mutation to amplification by a PCR utilizing a DNA polymerase without 3′→5′ exonuclease activity in the presence of a detection primer and a second primer, wherein 3′ end of the detection primer is complementary to a mutated base on a first DNA strand of the B-RAF gene and the second primer is complementary to a segment of the opposite DNA strand of the B-RAF gene and selected such that a detectable amplification product will be produced if the PCR occurs; and

(b) detecting whether the DNA segment is amplified.

Preferably, the 3′ end of the detection primer is complementary to the mutated base and significant amplification takes place only when the mutation is present. Thus, the present invention further relates to a method for detecting a specific mutation in the B-RAF gene, which comprises:

(a) subjecting a segment of the B-RAF gene containing the mutation to amplification by a PCR utilizing a DNA polymerase without exonuclease activity in the presence of a detection primer and a second primer, wherein 3′ end of the detection primer is complementary to a mutated base on a first DNA strand of the B-RAF gene and the second primer is complementary to a segment of the opposite DNA strand of the B-RAF gene and selected such that a detectable amplification product will be produced if the PCR occurs;

(b) detecting the presence or absence of the amplification product; and

(c) indicating that the specific mutation is present when the segment of the B-RAF gene is amplified.

Table 1 depicts oligonucleotide segments that are useful as the 3′ end of detection primer according to the inventive method for detecting the specified B-RAF mutations.

TABLE 1

| | | | |
|---|---|---|---|
| | Detection primer | | |
| | oligonucleotide | B-RAF | protein |
| SEQ ID No. | segment (5′→3′) | mutation | change |

| | | | |
|---|---|---|---|
| 1 | GGACAAAGAATTGA | G1388A | G463E |
| 2 | GGACAAAGAATTGT | G1388T | G463V |
| 3 | AGAATTGGATCTGC | G1394C | G465A |
| 4 | AGAATTGGATCTGA | G1394A | G465E |
| 5 | AGAATTGGATCTGT | G1394T | G465V |
| 6 | TGTGGATCATTTGC | G1403C | G468A |
| 7 | TCTGGATCATTTGA | G1403A | G468E |
| 8 | TATATTTCTTCATA | G1753A | E585K |
| 9 | AAATAGGTGATTTG | T1782G | F594L |
| 10 | AATAGGTGATTTTC | G1783C | G595R |
| 11 | AGGTGATTTTGGTG | C1786G | L596V |
| 12 | GGTGATTTTGGTCG | T1787G | L596R |
| 13 | GGTCTAGCTACAGA | T1796A | V599E |
| 14 | GTCTAGCTACAGAT | TG1796-97AT | V599D |

As indicated above, the primer should comprise at least about 14 nucleotides and are preferably comprises 16-24 nucleotides. Thus, the primer should comprise the 14 or so nucleotides specified in Table 1 on its 3′ end. However, useful primers often contain additional nucleotides at the 5′ end. It is only important that the oligonucleotide contain sufficient nucleotides to function as a primer and that the nucleotide at the 3′ end be known to be complimentary to the mutation.

Thus, the present invention relates to the a method for detecting a G1388A mutation in a human B-RAF gene, which comprises:

(a) subjecting a segment of the B-RAF gene containing the G1388A mutation to amplification by a PCR in the presence of a detection primer which comprises a 3′ end having SEQ ID No. 1 and a second primer, wherein the second primer is complementary to a segment of the opposite DNA strand of the B-RAF gene and selected such that a detectable amplification product will be produced if the PCR occurs;

(b) detecting whether the DNA segment is amplified; and

(c) indicating that the specific mutation is present when the segment of the B-RAF gene is amplified.

The present invention further relates to the a method for detecting a G1388T mutation in a human B-RAF gene, which comprises:

(a) subjecting a segment of the B-RAF gene containing the G1388T mutation to amplification by a PCR in the presence of a detection primer which comprises a 3′ end having SEQ ID No. 2 and a second primer, wherein the second primer is complementary to a segment of the opposite DNA strand of the B-RAF gene and selected such that a detectable amplification product will be produced if the PCR occurs;

(b) detecting whether the DNA segment is amplified; and

(c) indicating that the specific mutation is present when the segment of the B-RAF gene is amplified.

The present invention further relates to the a method for detecting a G1394C mutation in a human B-RAF gene, which comprises:

(a) subjecting a segment of the B-RAF gene containing the G1394C mutation to amplification by a PCR in the presence of a detection primer which comprises a 3′ end having SEQ ID No. 3 and a second primer, wherein the second primer is complementary to a segment of the opposite DNA strand of the B-RAF gene and selected such that a detectable amplification product will be produced if the PCR occurs;

(b) detecting whether the DNA segment is amplified; and

(c) indicating that the specific mutation is present when the segment of the B-RAF gene is amplified.

The present invention further relates to the a method for detecting a G1394A mutation in a human B-RAF gene, which comprises:

(a) subjecting a segment of the B-RAF gene containing the G1394A mutation to amplification by a PCR in the presence of a detection primer which comprises a 3′ end having SEQ ID No. 4 and a second primer, wherein the second primer is complementary to a segment of the opposite DNA strand of the B-RAF gene and selected such that a detectable amplification product will be produced if the PCR occurs;

(b) detecting whether the DNA segment is amplified; and

(c) indicating that the specific mutation is present when the segment of the B-RAF gene is amplified.

The present invention further relates to the a method for detecting a G1394T mutation in a human B-RAF gene, which comprises:

(a) subjecting a segment of the B-RAF gene containing the G1394T mutation to amplification by a PCR in the presence of a detection primer which comprises a 3′ end having SEQ ID No.5 and a second primer, wherein the second primer is complementary to a segment of the opposite DNA strand of the B-RAF gene and selected such that a detectable amplification product will be produced if the PCR occurs;

(b) detecting whether the DNA segment is amplified; and

(c) indicating that the specific mutation is present when the segment of the B-RAF gene is amplified.

The present invention further relates to the a method for detecting a G1403C mutation in a human B-RAF gene, which comprises:

(a) subjecting a segment of the B-RAF gene containing the G1403C mutation to amplification by a PCR in the presence of a detection primer which comprises a 3′ end having SEQ ID No. 6 and a second primer, wherein the second primer is complementary to a segment of the opposite DNA strand of the B-RAF gene and selected such that a detectable amplification product will be produced if the PCR occurs;

(b) detecting whether the DNA segment is amplified; and

(c) indicating that the specific mutation is present when the segment of the B-RAF gene is amplified.

The present invention further relates to the a method for detecting a G1403A mutation in a human B-RAF gene, which comprises:

(a) subjecting a segment of the B-RAF gene containing the G1403A mutation to amplification by a PCR in the presence of a detection primer which comprises a 3′ end having SEQ ID No. 7 and a second primer, wherein the second primer is complementary to a segment of the opposite DNA strand of the B-RAF gene and selected such that a detectable amplification product will be produced if the PCR occurs;

(b) detecting whether the DNA segment is amplified; and

(c) indicating that the specific mutation is present when the segment of the B-RAF gene is amplified.

The present invention further relates to the a method for detecting a G1753A mutation in a human B-RAF gene, which comprises:

(a) subjecting a segment of the B-RAF gene containing the G1753A mutation to amplification by a PCR in the presence of a detection primer which comprises a 3′ end having SEQ ID No. 8 and a second primer, wherein the second primer is complementary to a segment of the opposite DNA strand of the B-RAF gene and selected such that a detectable amplification product will be produced if the PCR occurs;

(b) detecting whether the DNA segment is amplified; and

(c) indicating that the specific mutation is present when the segment of the B-RAF gene is amplified.

The present invention further relates to the a method for detecting a T1782G mutation in a human B-RAF gene, which comprises:

(a) subjecting a segment of the B-RAF gene containing the T1782G mutation to amplification by a PCR in the presence of a detection primer which comprises a 3′ end having SEQ ID No. 9 and a second primer, wherein the second primer is complementary to a segment of the opposite DNA strand of the B-RAF gene and selected such that a detectable amplification product will be produced if the PCR occurs;

(b) detecting whether the DNA segment is amplified; and

(c) indicating that the specific mutation is present when the segment of the B-RAF gene is amplified.

The present invention further relates to the a method for detecting a G1783C mutation in a human B-RAF gene, which comprises:

(a) subjecting a segment of the B-RAF gene containing the G1783C mutation to amplification by a PCR in the presence of a detection primer which comprises a 3′ end having SEQ ID No. 10 and a second primer, wherein the second primer is complementary to a segment of the opposite DNA strand of the B-RAF gene and selected such that a detectable amplification product will be produced if the PCR occurs;

(b) detecting whether the DNA segment is amplified; and

(c) indicating that the specific mutation is present when the segment of the B-RAF gene is amplified.

The present invention further relates to the a method for detecting a T1787G mutation in a human B-RAF gene, which comprises:

(a) subjecting a segment of the B-RAF gene containing the T1787G mutation to amplification by a PCR in the presence of a detection primer which comprises a 3′ end having SEQ ID No. 12 and a second primer, wherein the second primer is complementary to a segment of the opposite DNA strand of the B-RAF gene and selected such that a detectable amplification product will be produced if the PCR occurs;

(b) detecting whether the DNA segment is amplified; and

(c) indicating that the specific mutation is present when the segment of the B-RAF gene is amplified.

The present invention further relates to the a method for detecting a T1796A mutation in a human B-RAF gene, which comprises:

(a) subjecting a segment of the B-RAF gene containing the T1796A mutation to amplification by a PCR in the presence of a detection primer which comprises a 3′ end having SEQ ID No. 13 and a second primer, wherein the second primer is complementary to a segment of the opposite DNA strand of the B-RAF gene and selected such that a detectable amplification product will be produced if the PCR occurs;

(b) detecting whether the DNA segment is amplified; and

(c) indicating that the specific mutation is present when the segment of the B-RAF gene is amplified.

The present invention further relates to the a method for detecting a TG1796-97AT mutation in a human B-RAF gene, which comprises:

(a) subjecting a segment of the B-RAF gene containing the TG1796-97AT mutation to amplification by a PCR in the presence of a detection primer which comprises a 3′ end having SEQ ID No. 14 and a second primer, wherein the second primer is complementary to a segment of the opposite DNA strand of the B-RAF gene and selected such that a detectable amplification product will be produced if the PCR occurs;

(b) detecting whether the DNA segment is amplified; and

(c) indicating that the specific mutation is present when the segment of the B-RAF gene is amplified.

In a preferred embodiment, the detection primer comprises a 3′ end having one of SEQ ID Nos. 15-28 as depicted in Table 2.

TABLE 2

| | Detection primer | | |
|---|---|---|---|
| | oligonucleotide | B-RAF | Protein |
| SEQ ID No. | segment (5′→3′) | mutation | change |
| 15 | TGGGACAAAGAATTGA | G1388A | G463E |
| 16 | TGGGACAAAGAATTGT | G1388T | G463V |
| 17 | AAAGAATTGGATCTGC | G1394C | G465A |
| 18 | AAAGAATTGGATCTGA | G1394A | G465E |
| 19 | AAAGAATTGGATCTGT | G1394T | G465V |
| 20 | GATCTGGATCATTTGC | G1403C | G468A |
| 21 | GATCTGGATCATTTGA | G1403A | G468E |
| 22 | AATATATTTCTTCATA | G1753A | E585K |
| 23 | AAAAATAGGTGATTTG | T1782G | F594L |
| 24 | AAAATAGGTGATTTTC | G1783C | G595R |
| 25 | ATAGGTGATTTTGGTG | C1786G | L596V |
| 26 | TAGGTGATTTTGGTCG | T1787G | L596R |
| 27 | TTGGTCTAGCTACAGA | T1796A | V599E |
| 28 | TGGTCTAGCTACAGAT | TG1796-97AT | V599D |

In an especially preferred embodiment, the detection primer indicated in Table 3 is utilized to detect the corresponding mutation.

TABALE 3

| | Detection primer | | |
|---|---|---|---|
| SEQ ID | oligonucleotide | B-RAF | Protein |
| No. | segment (5′→3′) | mutation | change |
| 29 | ACAGTGGGACAAAGAATTGA | G1388A | G463E |
| 30 | ACAGTGGGACAAAGAATTGT | G1388T | G463V |
| 31 | GGACAAAGAATTGGATCTGC | G1394C | G465A |
| 32 | GGACAAAGAATTGGATCTGA | G1394A | G465E |
| 33 | GGACAAAGAATTGGATCTGT | G1394T | G465V |
| 34 | ATTGGATCTGGATCATTTGC | G1403C | G468A |
| 35 | ATTGGATCTGGATCATTTGA | G1403A | G468E |
| 36 | GAGTAATAATATATTTCTTCATA | G1753A | E585K |
| 37 | CAGTAAAAATAGGTGATTTG | T1782G | F594L |
| 38 | CAGTAAAAATAGGTGATTTTC | G1783C | G595R |
| 39 | GTAAAAATAGGTGATTTTGGTG | C1786G | L596V |
| 40 | GTAAAAATAGGTGATTTTGGTCG | T1787G | L596R |
| 41 | GATTTTGGTCTAGCTACAGA | T1796A | V599E |
| 42 | GATTTTGGTCTAGCTACAGAT | TG1796-97AT | V599D |

In accordance with this aspect of the present invention, the second primer is selected such that it is complementary to a segment of the opposite DNA strand and sufficiently upstream or downstream of the mutation such that if the PCR occurs, the amplified product will contain enough nucleotides to be readily detectable. Preferably, the second primer is selected such that it is at least about 14 nucleotides in length, and preferably about 16-24 nucleotides in length, for example, 20-24 nucleotides in length, the amplification product will contain at least about 70 base pairs, for example, 70-6,000 base pairs, preferably 100-1,000 base pairs, for example, 150-500 base pairs, and preferably has a melting point similar to that of the detection primer.

Thus, the oligonucleotides depicted in Table 4 are useful as the second primer for detecting the indicated B-RAF mutations in conjunction with the detection primers described above for the indicated B-RAF mutation.

TABLE 4

| | Second primer | |
|---|---|---|
| | oligonucleotide | |
| SEQ ID No. | segment (5′→3′) | B-RAF mutation |
| 43 | TGTCACCACATTACATACTTACC | G1388A |
| 44 | TGTCACCACATTACATACTTACC | G1388T |
| 45 | TGTCACCACATTACATACTTACC | G1394C |
| 46 | TGTCACCACATTACATACTTACC | G1394A |
| 47 | TGTCACCACATTACATACTTACC | G1394T |
| 48 | TGTCACCACATTACATACTTACC | G1403C |
| 49 | TGTCACCACATTACATACTTACC | G1403A |
| 50 | GACTTTCTAGTAACTCAGCAG | G1753A |
| 51 | GACTTTCTAGTAACTCAGCAG | T1782G |
| 52 | GACTTTCTAGTAACTCAGCAG | G1783C |
| 53 | GACTTTCTAGTAACTCAGCAG | C1786G |
| 54 | GACTTTCTAGTAACTCAGCAG | T1787G |
| 55 | GACTTTCTAGTAACTCAGCAG | T1796A |
| 56 | GACTTTCTAGTAACTCAGCAG | TG1796-97AT |

In an especially preferred embodiment of this aspect of the invention, the detection primer identified in Table 3 is utilized in conjunction with the second primer identified in Table 4 for the corresponding mutation.

Each detection primer listed in Tables 1, 2, 3 and 5 may be used according to the present invention for the detection of a corresponding B-RAF mutation as reported in the Tables. The present invention also pertains to the PCR method herein described using a detection primer for detecting the corresponding mutation as listed in the Tables 1, 2, 3 and 5.

TABLE 5

SEQ ID
No.

| | | | |
|---|---|---|---|
| | Detection primer oligonu- | | |
| | cleotide segment (5′→3′) | | |
| 57 | GGACCCACTCCATCGAGATTTCT | T1796-97A | V599E |
| 59 | GACCCACTCCATCGAGATTTCT | T1796-97A | V599E |
| 60 | ACCCACTCCATCGAGATTTCT | T1796-97A | V599E |
| 61 | CCCACTCCATCGAGATTTCT | T1796-97A | V599E |
| 62 | CCACTCCATCGAGATTTCT | T1796-97A | V599E |
| | Second primer oligonu- | | |
| | cleotide segment (5′→3′) | | |
| 58 | CATAATGCTTGCTCTGATAGG | T1796-97A | V599E |

The primer should comprise at least 14 nucleotides and preferably comprises 16-24 nucleotides, e.g. 23, 22, 21, 20, 19 nucleotides. According to the invention, useful primers derived from SEQ ID No. 57 can contain less nucleotides at the 5′ end and should comprise the 14 nucleotides specified in Table 5 on its 3′ end. It is important that the nucleotide contains sufficient nucleotides to function as a primer and that the nucleotide at the 3′ end be known to be complementary to the mutation.

In one other embodiment of the invention, the detection primer SEQ ID No.57 is used with the second primer SEQ ID No. 58 for detection of the mutation T1796-97A (V599E).

In a further embodiment of the invention, the detection primer SEQ ID No. 41 is used with the second primer SEQ ID No. 55 for detection of the mutation T1796-97A (V599E).

In another aspect, the present invention relates to oligonucleotide primers for PCR amplification of a mutated human B-RAF gene. Thus, the present invention includes oligonucleotides comprising SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13 or SEQ ID No. 14.

In a preferred embodiment of this aspect of the invention, the oligonucleotide primers comprises SEQ ID No. 15, SEQ ID No. 16, SEQ ID No. 17, SEQ ID No. 18, SEQ ID No. 19, SEQ ID No. 20, SEQ ID No. 21, SEQ ID No. 22, SEQ ID No. 23, SEQ ID No. 24, SEQ ID No. 25, SEQ ID No. 26, SEQ ID No. 27 or SEQ ID No. 28.

In a particularly preferred embodiment of this aspect of the invention, the oligonucleotide primers consist of SEQ ID No. 29, SEQ ID No. 30, SEQ ID No. 31, SEQ ID No. 32, SEQ ID No. 33, SEQ ID No. 34, SEQ ID No. 35, SEQ ID No. 36, SEQ ID No. 37, SEQ ID No. 38, SEQ ID No. 39, SEQ ID No. 40, SEQ ID No. 41 or SEQ ID No. 42.

In a further aspect, the invention pertains to the oligonucleotide primers having the SEQ ID Nos. 57, 59, 60, 61 and 62.

The following example is intended to further illustrate, but not further limit, the present invention.

### EXAMPLE 1

### Detection of T1796A Mutation in the Human B-RAF Gene

Detection primer: SEQ ID No. 41, Second primer: SEQ ID No. 55

Genomic DNA is isolated from human cells from a melanoma cell line using a GENELUTE mammalian genomic DNA kit (Sigma Cat. No. G1N 350). PCR reactions are carried out on a PCR machine (MJ Research, Model PTC100) in a total volume of 50 microL using the PCR Core kit by Roche (Cat. No. 1578 553). The PCR reaction micture contains 5 microL of 10× reaction buffer, 1 microL of 10 mM dNTPs, 100-1,000 ng of template DNA, 0.5 microL Taq polymerase (2.5-5 U), 1 microL of a 31 μM stock of each primer.

The PCR conditions are as follows: 95° C. for 3 min, 35 cycles of [94° C. for 1 min, 50° C. for 30 sec, 72° C. for 1 min], 72° C. for 10 min, followed by soaking at 4° C.

After amplification, 8 microLs of the PCR reaction mixture is mixed with 2 microLs of nucleic acid sample loading buffer [BioRad Cat. No. 161-0767]. The 10 microL sample is loaded onto a 1.5% agarose [GIBCO-BRL Cat. No. 15510-027] gel that contains 0.3 μg/mL of ethidium bromide [Pierce Cat. No. 17898]. Molecular weight standards [100 bp DNA ladder from Invitrogen Cat. No. 10380-012] are loaded in an adjacent lane. The DNA is separated by electrophoresis in TAE buffer (0.04 M Tris-acetate, 0.01 M EDTA. 0.02 M glacial acetic acid pH 8.4) [Roche Cat. No. 1666690]. Electrophoresis conditions are 120 volts for 30-40 minutes. After separation, the gel is exposed to UV light and a picture taken on a AlphaImager2000 documentation system.

Generally, two bands are detected in the gel. The faster migrating band runs ahead of the 100 bp marker and represents the primers. The DNA that results from the T1796A mutant specific PCR amplification has a predicted size of 152 bp and migrates between the 100 bp standard and the 200 bp standard as predicted. The PCR amplification product is confirmed by sequencing. The presence of the PCR amplification product demonstrates that the T1796A mutation is present in the template DNA.

### EXAMPLE 2

### Detection of T1796A Mutation in the Human B-RAF Gene

The method of example 1 was performed on genomic DNA isolated from human cells from a melanoma cell line with the detection primer SEQ ID No. 57 and the second primer SEQ ID No. 58.

The DNA that results from the T1796A mutant specific PCR amplification has a predicted size of 142 bp and migrates between the 100 bp standard and the 200 bp standard as predicted. The PCR amplification product is confirmed by sequencing. The presence of the PCR amplification product demonstrates that the T1796A mutation is present in the template DNA.

### EXAMPLE 3

### Detection of T1796A Mutation in the Human B-RAF Gene from DNA samples from Patients with Melanoma and Colon Cancers

Genomic DNA from 100 patients with melanoma and 100 patients with colon cancer was analyzed according to the methods described in Example 1 and Example 2. The method of Example 1 with the primers SEQ ID No. 41 and SEQ ID No. 55 gives the same results as the method in Example 2 with the primers SEQ ID No. 57 and SEQ ID No. 58.

62.9 % of the patients with melanoma cancers and 8.2% of the patients with colon cancers have the V599E mutation.

1. A method for detecting a specific mutation in the B-RAF gene, which comprises:
(a) subjecting a segment of the B-RAF gene containing the mutation to amplification by a PCR utilizing a DNA polymerase without 3′→5′ exonuclease activity in the presence of a detection primer and a second primer, wherein 3′ end of the detection primer is complementary to a mutated base on a first DNA strand of the B-RAF gene and the second primer is complementary to a segment of the opposite DNA strand of the B-RAF gene and selected such that a detectable amplification product will be produced if the PCR occurs; and
(b) detecting whether the DNA segment is amplified.

2. A method of claim 1, wherein the detection primer comprises SEQ ID Nos. 1, 2, 3, 4, 5, 6, 7, 8, 9, 10, 11, 12, 13 or 14.

3. An oligonucleotide primer comprising SEQ ID Nos. 1, 2, 3, 4, 5, 6, 7, 8, 9, 10, 11, 12, 13 or 14.

4. An oligonucleotide primer comprising SEQ ID Nos. 15, 16, 17, 18, 19, 20, 21, 22, 23, 24, 25, 26, 27 or 28.

5. An oligonucleotide primer comprising SEQ ID Nos. 29, 30, 31, 32, 33, 34, 35, 36, 37, 38, 39, 40, 41 or 42.

6. An oligonucleotide primer comprising SEQ ID Nos. 57, 59, 60, 61 or 62.

7. An oligonucleotide primer for use as a detection primer according to the method of claim 1 wherein said primer comprises SEQ ID Nos. 15, 16, 17, 18, 19, 20, 21, 22, 23, 24, 25, 26, 27 or 28.

8. An oligonucleotide primer for use as a detection primer according to the method of claim 1 wherein said primer comprises SEQ ID Nos. 29, 30, 31, 32, 33, 34, 35, 36, 37, 38, 39, 40, 41 or 42.

9. An oligonucleotide primer for use as a detection primer according to the method of claim 1 wherein said primer comprises SEQ ID Nos. 57, 59, 60, 61 or 62.
